(19) United States
(12) Patent Application Publication
(10) Pub. No.: US 2023/0210783 A1
(43) Pub. Date: Jul. 6, 2023

(54) NANOSPHERE SIZE CONTROL BY VARYING THE RATIO OF COPOLYMER BLENDS

(71) Applicant: Rutgers, The State University of New Jersey, New Brunswick, NJ (US)

(72) Inventors: Joachim B. Kohn, Piscataway, NJ (US); Mariana Reis Nogueira de Lima, Chacara Santo Antonio (BR)

(73) Assignee: Rutgers, The State University of New Jersey, New Brunswick, NJ (US)

(21) Appl. No.: 18/001,398

(22) PCT Filed: Jun. 11, 2021
(86) PCT No.: PCT/US21/36997

§ 371 Date: Dec. 9, 2022

Related U.S. Application Data
(60) Provisional application No. 63/038,323, filed on Jun. 12, 2020.

(51) Int. Cl. A61K 9/51; C08G 63/13; A61K 45/06

(52) U.S. Cl. CPC A61K 9/5192; C08G 63/13; A61K 45/06; C08G 2230/00; B82Y 5/00

(57) ABSTRACT

Nanosphere composition containing a mixture of a triblock oligomer and a diblock oligomer for the delivery of an active agent. Also disclosed are methods of preparing the nanospheres and methods of delivering an active agent enclosed in the nanospheres.

### CROSS REFERENCE TO RELATED APPLICATIONS

This application claims the benefit of U.S. provisional patent application No. 63/038,323 filed on Jun. 12, 2020, the content of which is hereby incorporated by reference herein.

### FIELD OF THE INVENTION

The present disclosure relates to nanospheres comprising a mixture of a triblock oligomer and a diblock oligomer for the delivery of an active agent.

### BACKGROUND OF THE INVENTION

Synthetic polymers have shown advantages as delivery vehicles for many pharmacological materials, providing increased solubility and stability of bound therapeutic compounds and the opportunity for targeted delivery to a restricted population of cells. Nanospheres, i.e., carriers with a size in the submicron range, are desirable for intravascular administration. For this purpose, the recent advances in supramolecular chemistry allow designing materials of superior characteristics.

For parenteral delivery systems, it has been shown that nano-sized particles and liposomes have great potential in cancer therapy due to their ability to extravasate from the leaky vasculature of tumors. To achieve this objective, various nano-sized particles or colloidal carriers such as nanospheres, polymeric micelles, liposomes, and surface modified nanoparticles have been proposed. However, the distribution of drugs and carriers in the body, undesirable side effects, rapid clearance by macrophage, thermal instability, structural fragility and low drug loading efficiency, among other factors, have limited these approaches and only a few such delivery systems have progressed toward clinical use.

Of particular interest are nanoparticles formed via the self-assembly of block copolymers. Similar to low molecular weight lipid or surfactant molecules, amphiphilic block copolymers consist of at least two parts, a hydrophilic block and a hydrophobic block. Such amphiphilic block copolymers, driven by their hydrophobicity, can self-assemble in aqueous solution. At high concentrations, they may build lamellar liquid crystalline phases whereas, in dilute aqueous solution, they may form superstructures of various shapes like micelles or vesicular structures.

A suitable neutral amphiphilic block copolymer forms spontaneously nanometer-sized, well-defined hollow sphere structures in dilute aqueous solution. These structures can be viewed as the high molecular weight analogues of lipid or surfactant molecules. However due to their slow dynamic, they form much more stable superstructures than conventional liposomes. Furthermore, liposomes, e.g., spherically closed lipid bilayers, are rapidly recognized by the immune system and cleared from the blood stream. Due to the wide variety of block copolymer chemistry one can prepare an entirely synthetic material to avoid immunogenic reactions.

Although it is well known that suitable block copolymers can form nanospheres, few were designed to self-assemble into biocompatible and biodegradable structures in dilute aqueous solution. One example of spontaneous aggregation of an amphiphilic block oligomer has been reported with a poly(methyloxazoline)-block-poly(dimethyl-siloxane)-block-poly (methyl-oxazoline), PMOXA-PDMS-PMOXA triblock oligomer. Injection combined with extrusion techniques leads to the formation of vesicles whose size can be controlled between 50 and 500 nm. U.S. Pat. No. 8,591,951 provides a biocompatible non-toxic triblock copolymers having an A-B-A structure wherein each A is a hydrophilic, biocompatible end block and the B middle block is a hydrophobic desaminotyrosyl tyrosine polycarbonate or polyarylate. The copolymers spontaneously self-assemble to form low critical aggregation concentration nanospheres having utility as delivery vehicles for hydrophobic biologically or pharmaceutically active compounds. However, there remains a need for non-cytotoxic, biodegradable copolymer vesicles, especially those with suitable size for targeted delivery.

### SUMMARY OF THE INVENTION

This patent document provides nanospheres of suitable size for delivery of an active agent in various applications. Size is an important parameter that can be used to optimize the performance of nanospheres in vivo and to increase their efficiency for therapeutic and cosmetic applications. Of particular interest is the use of the nanospheres in passively targeting therapeutic or diagnostic agents to specific biological environments via suitable means of administration.

An aspect of the patent document provides a nanosphere composition for delivery of an active agent. The composition includes a distribution of nanospheres with essentially the same hydrodynamic Z-average diameter in a pharmaceutically acceptable carrier, wherein the nansospheres consisting essentially of a mixture of the same triblock oligomer and the same diblock oligomer. The triblock oligomer consists of a single A-B-A structure and the diblock oligomer consists of a single A-B-H structure, wherein A and B in the diblock oligomer are identical to A and B in the triblock oligomer, wherein the B block is hydrophobic with the same or different repeating units having the structure according to Formula I:

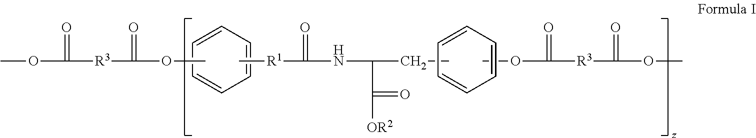

wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms, wherein R² and R³ together contain more than 6 carbons, provided that when R² is (CH₂)₃CH₃, R³ is not (CH₂)₄;
each A block is a poly(alkylene oxide) having the structure:

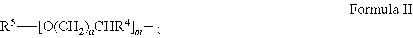

R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
m for each A is independently selected to provide a molecular weight for each A between about 1000 and about 15,000 g/mol.

In some embodiments, the A block has the structure CH₃O—[CH₂—CH₂—O—]ₘ.

In some embodiments, R¹ is —CH₂—CH₂—. In some embodiments, R² is selected from the group consisting of ethyl, butyl, hexyl, octyl, decyl, dodecyl and benzyl groups. In some embodiments, R³ contains up to 12 carbon atoms. In some embodiments, R³ is selected from the group consisting of —CH₂—CH₂—C(═O)—, —CH═CH—, —CH₂—CH(—OH)—, —CH₂—C(═O)— and (—CH₂—)_{Y}, wherein Y is between 0 and 12, inclusive.

In some embodiments, the diblock oligomer is at least 30% of the total weight of the diblock oligomer and the triblock oligomer. In some embodiments, the diblock oligomer ranges from about 40% to about 99% of the total weight of the diblock oligomer and the triblock oligomer.

In some embodiments, the nanospheres enclose a pharmaceutically active hydrophobic compound. In some embodiments, the pharmaceutically active hydrophobic compound is selected from the group consisting of anti-tumor agents, antibiotics, antimicrobials, statins, peptides, proteins, hormones, and vaccines. In some embodiments, the hydrophobic compound is selected from the group consisting of paclitaxel, camptothecin, 9-nitrocamptothecin, cisplatin, carboplatin, ciprofloxacin, doxorubicin, rolipram, simvastatin, methotrexate, indomethacin, probiprofen, ketoprofen, iroxicam, diclofenac, cyclosporine, etraconazole, rapamycin, nocodazole, colchicine, ketoconazole, tetracycline, minocycline, doxycycline, ofloxacin, gentamicin, octreotide, calcitonin, interferon, testosterone, progesterone, estradiol, estrogen, and insulin. In some embodiments, the nanospheres enclose a contrast agent.

Another aspect of the patent document provides a composition comprising the nanospheres disclosed herein.

Another aspect provides a method of preparing nanospheres having a predetermined hydrodynamic Z-average diameter as measured by DLS. The nanospheres consists essentially of triblock oligomers having the same A-B-A structure and diblock oligomers having the same A-B-H structure, wherein A and B in the diblock oligomer are identical to A and B in the triblock oligomer. The method includes:
(a) blending separate quantities of the triblock and diblock oligomers, wherein the respective quantities are selected to provide the nanoparticles having a predetermined hydrodynamic Z-average diameter,
(b) dissolving the blended oligomers in an organic solvent in which the oligomers are soluble to provide an organic solution of the diblock and triblock oligomers, and
(c) adding the organic solution to an aqueous solution to form an aqueous suspension of the nanoparticles having a predetermined hydrodynamic Z-average diameter; wherein the B block is hydrophobic with repeating units having the structure according to Formula I:

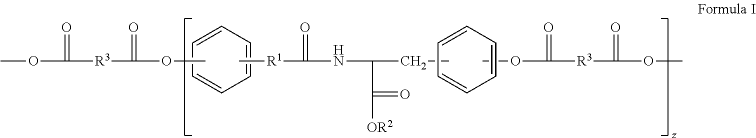

wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms, wherein R² and R³ together contain more than 6 carbons;
wherein the A block is a poly(alkylene oxide) having the structure:

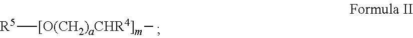

R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
m for each A is independently selected to provide a molecular weight for each A between about 1000 and about 15,000 g/mol.

In some embodiments, the diblock oligomer is about 5%, about 10%, about 20%, about 30%, about 40%, about 50%, about 60%, about 70%, about 80%, about 90%, or about about 95% in the total weight of the triblock oligomer and the diblock oligomer. In some embodiments, the average hydrodynamic Z-average diameter of the nanospheres range from about 35 nm to about 130 nm, from about 40 nm to about 120 nm, from about 50 nm to about 110 nm, from about 50 nm to about 100 nm. In some embodiments, the hydrodynamic Z-average diameter of the nanospheres is about 35 nm, about 40 nm, about 50 nm, about 60 nm, about 70 nm, about 80 nm, about 90 nm, about 100 nm, about 110 nm, or about 120 nm.

In some embodiments, the method further includes mixing a hydrophobic compound with the triblock oligomer and the diblock oligomer. In some embodiments, the hydrophobic compound is selected from the group consisting of anti-tumor agents, antibiotics, antimicrobials, statins, peptides, proteins, hormones, and vaccines.

Another aspect provides a method for site-specific or systemic drug delivery comprising administering to a subject in need thereof the nanosphere composition described herein.

Another aspect provides a diblock oligomer consisting of a single A-B-H structure wherein A and B are as defined above.

Another aspect provides a method of preparing the diblock oligomer of claim 20, comprising reacting Intermediate I-A with Intermediate I-B. In some embodiments, I-A is about 0.5 equivalent of total carboxylic acids present in I-B.

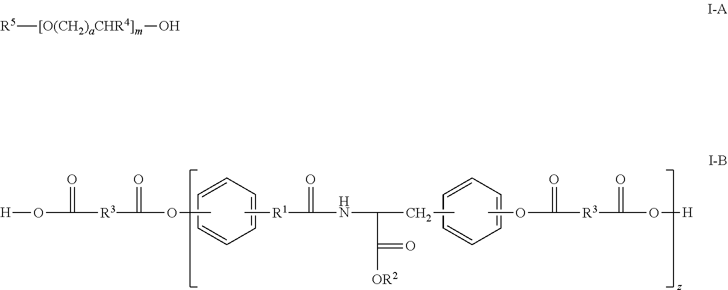

### BRIEF DESCRIPTION OF THE DRAWINGS

FIGS. 1(a), 1(b) and 1(c) illustrate synthesis of oligomers. FIG. 1(a) Reaction scheme of tyrosine-derived Oligo(DTO-SA) synthesis (B-block), and amphiphilic copolymers synthesis FIG. 1(b) Triblock (A-B-A), and FIG. 1(c) Diblock (A-B-H)). 1) DTO: desaminotyrosyl-tyrosine octyl ester; 2) SA: suberic acid; 3) Oligo(DTO-SA): Oligo(desaminotyrosyl-tyrosine octyl ester suberate); 4) Triblock (A-B-A) copolymer; 5) Diblock copolymer (A-B-H).

FIG. 2 illustrates mole fractions of a certain polymer (Triblock, (A-B-A), Diblock (A-B-H), oligo(DTO-SA) and mPEG(5k)) that could be outside their respective number-average molecular weight range (Mn±tSₙ) obtained by THF-GPC.

FIGS. 3(a) and 3(b) illustrate THF-GPC chromatograms of different compositions. FIG. 3(a) Overlay of final THF-GPC chromatograms of Triblock (A-B-A), Diblock (A-B-H), Oligo(DTO-SA) and mPEG(5k) (1 mg/mL); and FIG. 3(b) THF-GPC chromatogram of triblock, diblock, oligo(DTO-SA) and mPEG(5k) pre-mixed in THF with individual concentration of 0.5 mg/mL.

FIG. 4 illustrates ¹H-NMR spectrum (500 MHz, DMSO-d6) of a) Triblock (A-B-A) copolymer, b) Diblock (A-B-H) copolymer, c) Oligo(DTO-SA), d) mPEG(5k), e) SA: suberic acid, f) DTO: desaminotyrosyl-tyrosine octyl ester.

FIGS. 5(a), 5(b) and 5(c) illustrate Nanospheres size distribution obtained by dynamic light scattering (DLS) of some of the prepared nanospheres compositions: FIG. 5(a) 100% Triblock (Z-average hydrodynamic diameter=32.8±0.7 nm); FIG. 5(b) 25% Diblock:75% Triblock (Z-average hydrodynamic diameter=67.8±1.3. nm); FIG. 5(c) 100% Diblock (Z-average hydrodynamic diameter=129.3±2.3 nm). The number after the ±sign represents the standard deviation of three repetitions of the nanosphere preparation using one single polymer badge as the starting material.

FIG. 6 illustrates correlation between the % diblock in the copolymer blend composition (A-B-H diblock: A-B-A triblock) and the Z-average hydrodynamic diameter of nanospheres.

FIGS. 7(a) and 7(b) illustrate correlation between the % mPEG(5k) in the polymeric blend composition and the Z-average hydrodynamic diameter of nanospheres. FIG. 7(a) Blend composed of mPEG(5k) and Triblock copolymer (A-B-A); FIG. 7(b) Blend composed of mPEG(5k) and Diblock copolymer (A-B-H).

FIGS. 8(a) and 8(b) illustrate correlation between the % Oligo(DTO-SA) in the polymeric blend composition and the Z-average hydrodynamic diameter of nanospheres. FIG. 8(a) Blend composed of Oligo(DTO-SA) and Triblock copolymer (A-B-A); FIG. 8(b) Blend composed of Oligo(DTO-SA) and Diblock copolymer (A-B-H).

### DETAILED DESCRIPTION OF THE INVENTION

Various embodiments of this patent document provides nanospheres comprising a coassembly of two copolymers. By adjusting the ratio of the two copolymers, the size of the resulting nanospheres can be controlled. Because different nanosphere sizes allow different biological interactions, the technology disclosed herein finds applications in different fields including for example delivery of pharmaceutical agents, control of cellular responses in tissue engineering, and cosmetics applications for skin and personal care. Also, based on the non-toxicity and biodegradability of the self-assemble nanospheres they have utility in agriculture for delivery of high value nutrients, pesticides or insecticides.

Polymeric nanospheres are widely studied as drug delivery systems. Targeted delivery to specific tissues is a highly desirable goal to avoid systemic side effects in non-targeted tissues. Although nanosphere composition, surface chemistry, and shape affect tissue distribution, the most significant factor to influence cellular uptake and biodistribution is the size (diameter) of the nanospheres.

At the cellular level particle internalization rate and mechanism via endocytic pathways depends on size. For example, particles of −50 nm and −80 nm diameter favor Clathrin-Caveolin independent and Caveolin-dependent cell uptake pathways, respectively. Particles larger than 100 nm usually follow Clathrin-dependent mechanisms, macro-pinocytosis and phagocytosis.

Topical and transdermal administration are also affected by different nanoparticle sizes. Skin acts as a protective barrier against toxic substances and polymeric nanoparticles are commonly used as permeation enhancers. For instance, smaller nanospheres demonstrate more incorporation into the epidermal layer of hairy guinea pig skin than the larger nanospheres.

### Nanosphere Composition

An aspect this patent document provides a nanosphere composition comprising a distribution of nanospheres with essentially the same hydrodynamic Z-average diameter in a pharmaceutically acceptable carrier, said nansospheres consisting essentially of a mixture of the same triblock oligomer and the same diblock oligomer. By adjusting the ratio between the triblock oligomer and the diblock oligomer, the hydrodynamic Z-average diameter (measurable by dynamic light scattering technology (DLS)) can be controlled to suit the delivery of an active agent to a target location. The triblock oligomer consists of a single A-B-A structure and the diblock oligomer consists of a single A-B-H structure, wherein:

the B block is hydrophobic with the same or different repeating units having the structure according to the following formula:

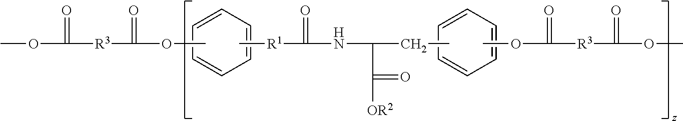

wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms, wherein R² and R³ together contain more than 6 carbons, provided that when R² is (CH₂)₃CH₃, R³ is not (CH₂)₄;
   each A block is a poly(alkylene oxide) having the structure,

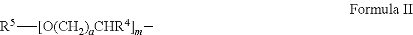

wherein
R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
   m is an integer ranging from 5 to 300, preferably for each A m is independently selected to provide a molecular weight for each A between about 1000 and about 15,000 g/mol.

In some embodiments, the A block has the structure:

CH₃O—[CH₂—CH₂—O—]ₘ.

In some embodiments, the subscript m ranges from 10 to 250, from 15 to 200, or from 50 to 150.

In some embodiments, R¹ is —CH₂—CH₂—. In some embodiments, the R¹ group can be further substituted with a C1-C4 alkyl.

In some embodiments, R² is selected from the group consisting of ethyl, butyl, hexyl, octyl, decyl, dodecyl and benzyl groups. In some embodiments, R² is selected from the group consisting of hexyl, octyl, decyl, and dodecyl groups.

In some embodiments, R³ contains up to 12 carbon atoms. In some embodiments, R³ contains 3, 4, 5, 6 or 7 carbon atoms.

In some embodiments, R² and R³ together include be more than 6, more than 8, or more than 12 carbons. In some embodiments, the carbons are in the form of methylene group.

In some embodiments, R³ is selected from —CH₂—CH₂—C(═O)—, —CH═CH—, —CH₂—CH(—OH)—, —CH₂—C(═O)— and (—CH₂—)_{Y}, wherein Y is between 0 and 12, inclusive.

By varying the ratio or relative weight percentage of the diblock oligomer and the triblock oligomer, the size of their nanosphere assembly can be adjusted. In some embodiments, the diblock oligomer is at least 2%, at least 3%, at least 5%, at least 10%, least 20%, at least 30%, least 40%, least 50%, least 60%, least 70%, least 80%, least 90% of the total weight of the diblock oligomer and the triblock oligomer. In some embodiments, the diblock oligomer ranges from about 2% to about 99%, from about 10% to about 99%, from about 20% to about 90%, from about 30% to about 85%, from about 40% to about 80%, from about 40% to about 70%, from about 40% to about 60%, from about 40% to about 50%, from about 20% to about 60%, from about 20% to about 50%, from about 20% to about 40%, or from about 30% to about 50% of the total weight of the diblock oligomer and the triblock oligomer.

The hydrodynamic Z-average diameter of the resulting nanosphere ranges from 35 nm to 130 nm, from 35 nm to 120 nm, from 35 nm to 100 nm, from 50 nm to 100 nm, from 50 nm to 80 nm, from 50 nm to 70 nm, from 40 nm to 80 nm, from 40 nm to 60 nm, from 80 nm to 120 nm, from 100 nm to 120 nm, from 35 nm to 45 nm, from 35 nm to 40 nm, from 40 nm to 43 nm, from 43 nm to 45 nm, from 48 nm to 68 nm, from 50 nm to 65 nm, from 50 nm to 60 nm, from 50 nm to 55 nm, from 55 nm to 75 nm, from 75 to 130 nm, from 75 nm to 125 nm, from 75 nm to 120 nm, from 75 nm to 110 nm, from 75 nm to 100 nm, 75 nm to 90 nm, 75 nm to 80 nm, from 80 nm to 100 nm, from 90 nm to 120 nm, from 90 nm to 110 nm, from 100 nm to 130 nm, from 110 nm to 130 nm, from 110 nm to 120 nm, or from 110 nm to 125 nm.

Depending on the target site or tissue, the size of nanospheres can be adjusted. In addition, the nanospheres may contain a pharmaceutically active agent or a diagnostic agent. Nonlimiting examples of the pharmaceutically active hydrophobic compound include anti-tumor agents, antibiotics, antimicrobials, statins, peptides, proteins, hormones, and vaccines. Additional examples of a hydrophobic compound as the pharmaceutically active agent include paclitaxel, camptothecin, 9-nitrocamptothecin, cisplatin, carboplatin, ciprofloxacin, doxorubicin, rolipram, simva-statin, methotrexate, indomethacin, probiprofen, ketoprofen, iroxicam, diclofenac, cyclosporine, etraconazole, rapamycin, nocodazole, colchicine, ketoconazole, tetracycline, minocycline, doxycycline, ofloxacin, gentamicin, octreotide, calcitonin, interferon, testosterone, progesterone, estradiol, estrogen, and insulin. In some embodiments, the nanospheres encloses a contrast agent for diagnostic purpose.

The nanospheres can also be used to co-deliver multiple agents, thereby resulting in their synergistic or additive effects and generally less required dosage for therapeutic efficacy. Synergy can be achieved by encapsulating an active agent and a secondary agent in the nanopsheres.

The composition can be used to deliver one or more therapeutic, prophylactic or diagnostic agents to an individual or subject in need thereof. The composition can be in the form of a liquid formulation, which includes the nanospheres disclosed herein in a liquid pharmaceutical carrier. Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Preferably, liquid formulations are isotonic relative to physiological fluids and of approximately the same pH, ranging from about pH 4.0 to about pH 7.4, more preferably from about pH 6.0 to pH 7.4. The liquid pharmaceutical carrier can include one or more physiologically compatible buffers, such as a phosphate. One skilled in the art can readily determine a suitable saline content and pH for an aqueous solution for pulmonary administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of assembled compositions to targeted tissues, e.g. through circulation.

The composition can be in the form of a dry solid/powder formulation and included in a kit. The dry powder formulation can be stored in separate containers, or mixed at specific ratios and stored. In some embodiments, suitable aqueous and organic solvents are included in additional containers. In some embodiments, the dry powder formulation is included in a kit. Alternatively, stabilized nanospheres are dried via vacuum-drying or freeze-drying, and suitable pharmaceutical liquid carrier can be added to rehydrate and suspend the assembled nanospheres upon use. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride.

Intravenous delivery is a common route for nanosphere administration. In some embodiments, the composition is formulated for parenteral delivery, such as injection or infusion, in the form of a solution or suspension. The formulation can be administered via any route, such as, the blood stream or directly to the organ or tissue to be treated. For example, parenteral administration may include administration to a patient intravenously, intradermally, intraperitoneally, intrapleurally, intratracheally, intramuscularly, subcutaneously, subjunctivally, by injection, and by infusion. The nanospheres leave the circulatory system through fenestrations (openings of the endothelial barrier) reaching the extra vascular compartments. Since the fenestration size varies according to organs and to pathological conditions, localization of nanospheres in specific tissues depends on nanospheres size.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized gelators, stabilizing agents, and/or active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Preservatives can be used to prevent the growth of fungi and micro-organisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. These preferably are enteric coated to avoid disassembly when passing through the stomach.

Excipients, including plasticizers, pigments, colorants, stabilizing agents, and glidants, may also be used to form coated compositions for enteral administration. Formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules.

### Method of Preparation

Another aspect of the patent document provides a method of preparing nanoparticles having a predetermined hydrodynamic Z-average diameter as measured by DLS, consisting essentially of triblock oligomers having the same A-B-A structure and diblock oligomers having the same A-B-H structure, wherein A and B in the diblock oligomer are identical to A and B in the triblock oligomer.

The method includes:
(a) blending separate quantities of the triblock and diblock oligomers, wherein the respective quantities are selected to provide the nanoparticles having a predetermined hydrodynamic Z-average diameter,
(b) dissolving the blended oligomers in an organic solvent in which the oligomers are soluble to provide an organic solution of the diblock and triblock oligomers, and
(c) adding the organic solution to an aqueous solution to form an aqueous suspension of the nanoparticles having a predetermined hydrodynamic Z-average diameter; wherein the B block is hydrophobic with repeating units having the structure according to Formula I:

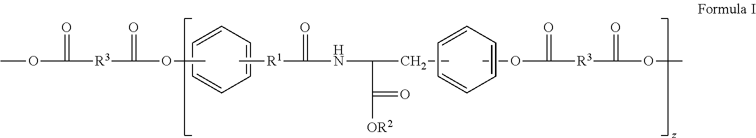

wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms;
wherein the A block is a poly(alkylene oxide) having the structure:

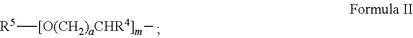

R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
m for each A is independently selected to provide a molecular weight for each A between about 1000 and about 15,000 g/mol.

In some embodiments of the method, R² and R³ together contain more than 6 carbons, more than 8 carbons or more than 12 carbons. In some embodiments, the carbons are in the form of methylene groups.

To ensure a suitable range of size for the nanospheres, the relative weight percentage of the triblock oligomer and the diblock oligomer need to be adjusted as described above. In some embodiments, the diblock oligomer ranges from about 2% to about 99%, from about 5% to about 99% in the total weight of the triblock oligomer and the diblock oligomer. In some embodiments, the diblock oligomer ranges from about 40% to about 90% in the total weight of the triblock oligomer and the diblock oligomer. In some embodiments, the diblock oligomer ranges from about 10% to about 99%, from about 20% to about 90%, from about 30% to about 85%, from about 40% to about 80%, from about 40% to about 70%, from about 40% to about 60%, from about 40% to about 50%, from about 20% to about 60%, from about 20% to about 50%, from about 20% to about 40%, or from about 30% to about 50% of the total weight of the diblock oligomer and the triblock oligomer.

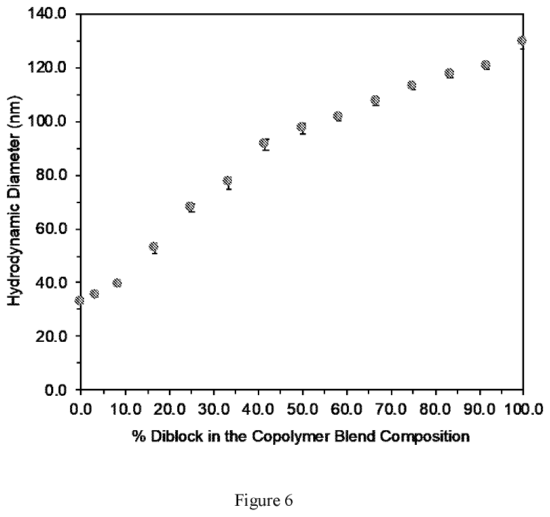

In order for the nanospheres to have a desirable range of size, a reference or a known standard (see for example FIG. 6) can be used. The reference shows the relationship between the size of the nanospheres and the percentage weight of an individual oligomer so that the mixing of the triblock and the diblock oligomers can performed under a predetermined manner. In some embodiments, the percentage of the diblock oligomer is so selected that the average hydrodynamic Z-average diameter of the nanospheres range from about 35 nm to about 130 nm. In some embodiments, the percentage of the diblock oligomer is so selected that the average hydrodynamic Z-average diameter of the nanospheres range from about 40 nm to about 120 nm. Other ranges of the size are as described above.

The preparation of the nanospheres may also be associated with the target site of tissue for delivery of an agent. For instance, nanospheres for uptake in lung and bone marrow tissues are generally smaller than nanospheres for liver and spleen uptake.

In some embodiments, the method further includes mixing an active agent with the triblock and the diblock oligomers. Upon formation of nanospheres, the agent will be encapsulated. In an example embodiment of encapsulating an active agent, the agent and the polymers are mixed in a solution. After stirring and centriguging the resulting mixture for a suitable period of time, the supernatant is discarded. The pellet can be washed and resuspended in a suitable solvent. The scope of the active agent is as described above.

### Method of Delivery of an Active Agent

Another aspect of the patent document provides a method for site-specific or systemic delivery of an active agent. The method generally includes administering to a subject in need thereof the nanosphere composition descried herein.

A typical dosage might range from about 0.001 mg/kg to about 1000 mg/kg, preferably from about 0.01 mg/kg to about 100 mg/kg, and more preferably from about 0.10 mg/kg to about 20 mg/kg. Advantageously, the nanospheres or a composition thereof may be administered several times daily, and other dosage regimens may also be useful.

The nanospheres or a composition thereof may be administered subcutaneously, intramuscularly, colonically, rectally, nasally, orally or intraperitoneally, employing a variety of dosage forms such as suppositories, implanted pellets or small cylinders, aerosols, oral dosage formulations and topical formulations, such as ointments, drops and transdermal patches. The dosage forms may optionally include one or more carriers.

Nanospheres encapsulating a hydrophobic agent to be delivered may also be dispersed as a reservoir of the agent within the oligomeric matrix of controlled release device. The host oligomeric matrix may be a hydrogel or other bioerodible oligomer. Such dispersions would have utility, for example, as active agent depots in transdermal drug delivery devices.

### Diblock Oligomer and Method of Preparation

Another aspect of the patent document provides a method of preparing the diblock oligomer. The method includes reacting Intermediate I-A with Intermediate I-B, wherein the amount of I-A ranges from about 0.2 to about 0.6 equivalent of available endgroups of I-B. Preferably, the amount of I-A is exactly 0.5 equivalents based on the calculation of the available end groups of I-B. Besides procedures illustrated in the examples disclosed herein, the preparation of the polyarylate oligomer and its coupling to PEG can be performed according to U.S. Pat. No. 8,591,951, which is incorporated by reference herein.

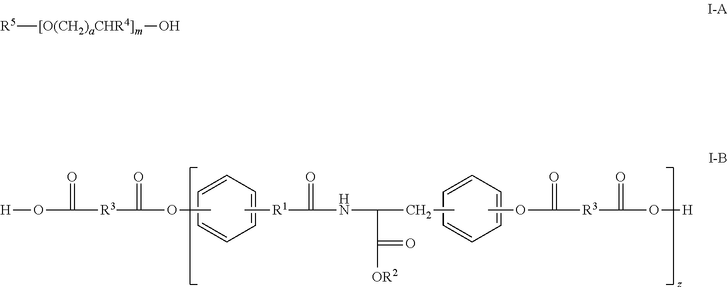

The diblock oligomer can be purified by various means known in the art. The purity of an oligomer can be determined by instruments available in the field such as gel permeation chromatography (GPC). In some embodiments, the isolated diblock oligomer has a purity of more than 70% or more than 95%.

### Definitions

The terms “alkyl”, “alkylene” and similar terms have the usual meaning known to those skilled in the art and thus may be used to refer to straight or branched hydrocarbon chain fully saturated (no double or triple bonds) hydrocarbon group. Terminal alkyl groups, e.g., of the general formula —CₙH₂ₙ₊₁, may be referred to herein as “alkyl” groups, whereas linking alkyl groups, e.g., of the general formula —(CH₂)ₙ—, may be referred to herein as “alkylene” groups. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like. A C₁-C₁₈ alkyl contains 1, 2, 3, 4, 5, 6, 7, 8, 9, 10, 11, 12, 13, 14, 15, 16, 17 or 18 carbons.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substitutent may be substituted with one of the above substituents.

An “alkylaryl” is an aryl group connected, as a substituent, via an alkylene group. The alkylene and aryl group of an aralkyl may be substituted or unsubstituted. Examples include but are not limited to benzyl, substituted benzyl, 2-phenylethyl, 3-phenylpropyl, and naphtylalkyl. In some cases, the alkylene group is a lower alkylene group. An alkylaryl group may be substituted or unsubstituted.

As noted above, alkyl groups may link together other groups, and in that context may be referred to as alkylene groups. Alkylene groups are thus biradical tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—(CH₂)₄—) groups. An alkylene group may be substituted or unsubstituted.

The terms “alkenyl”, “alkenylene” and similar terms have the usual meaning known to those skilled in the art and thus may be used to refer to an alkyl or alkylene group that contains in the straight or branched hydrocarbon chain containing one or more double bonds. An alkenyl group may be unsubstituted or substituted. When substituted, the substituent(s) may be selected from the same groups disclosed above with regard to alkyl group substitution unless otherwise indicated.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalo-methanesulfonamido, and amino, including mono- and di-substituted amino groups, and the protected derivatives thereof. When substituted, substituents on an aryl group may form a non-aromatic ring fused to the aryl group, including a cycloalkyl, cycloalkenyl, cycloalkynyl, and heterocyclyl.

As used herein, “heteroalkyl” refers to an alkyl group where one or more carbon atoms has been replaced with a heteroatom, that is, an element other than carbon, including but not limited to, nitrogen, oxygen and sulfur.

The terms “heteroalkyl”, “heteroalkylene,” and similar terms have the usual meaning known to those skilled in the art and thus may be used to refer to an alkyl group or alkylene group as described herein in which one or more of the carbons atoms in the backbone of alkyl group or alkylene group has been replaced by a heteroatom such as nitrogen, sulfur and/or oxygen. Likewise, the term “heteroalkenylene” may be used to refer to an alkenyl or alkenylene group in which one or more of the carbons atoms in the backbone of alkyl group or alkylene group has been replaced by a heteroatom such as nitrogen, sulfur and/or oxygen.

As used herein, “heteroaryl” refers to an aryl group where one or more carbon atoms has been replaced with a heteroatom, that is, an element other than carbon, including but not limited to, nitrogen, oxygen and sulfur.

For convenience and conciseness, sometimes the terms “alkyl”, “alkenyl”, “alkynyl”, “aryl”, “heteroaryl”, and “alkylaryl”, or the like, may be used to refer to the corresponding linking groups when they serve to connect two moieties of a molecule, either monomeric or polymeric, which should be readily understood by those skilled in the art. That is, on such occasions, “alkyl” should be interpreted as “alkylene”; “alkenyl” should be interpreted as “alkenylene”; “aryl” should be interpreted as “arylene”; and so on.

As used herein, the terms “polymer”, “polymeric” and similar terms have the usual meaning known to those skilled in the art and thus may be used to refer to homopolymers, copolymers (e.g., random copolymer, alternating copolymer, block copolymer, graft copolymer) and mixtures thereof. The repeating structural units of polymers may also be referred to herein as recurring units.

As used herein, the term “molecular weight” has the usual meaning known to those skilled in the art and thus reference herein to a polymer having a particular molecular weight will be understood as a reference to a polymer molecular weight in units of Daltons. Various techniques known to those skilled in the art, such as end group analysis (e.g., by ¹H NMR) and high-pressure size exclusion chromatography (HPSEC, also known as gel permeation chromatography, “GPC”), may be used to determine polymer molecular weights. In some cases, the molecular weights of polymers are further described herein using the terms “number average” molecular weight (Mn) and/or “weight average” molecular weight (Mw), both of which terms are likewise expressed in units of Daltons and have the usual meaning known to those skilled in the art.

Unless otherwise indicated, when a substituent is deemed to be “optionally substituted,” or “substituted” it is meant that the substituent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Similarly, the term “optionally ring-halogenated” may be used to refer to a group that optionally contains one or more (e.g., one, two, three or four) halogen substituents on the aryl and/or heteroaryl ring. The protecting groups that may form the protective derivatives of the above substituents are known to those of skill in the art and may be found in references such as Greene and Wuts, Protective Groups in Organic Synthesis, 3^{rd} Ed., John Wiley & Sons, New York, N.Y., 1999, which is hereby incorporated by reference in its entirety.

The Z-average diameter/size, also used as hydrodynamic Z-average diameter is the intensity weighted mean hydrodynamic diameter of the ensemble collection of nanoparticles measured by dynamic light scattering (DLS). Unless otherwise noted, hydrodynamic diameter refers to Z-average diameter.

Block copolymers of the present invention may be prepared according to the method disclosed in U.S. Pat. No. 8,591,951 and the references cited therein, the disclosure of all of which are hereby incorporated by reference. The poly(alkylene oxide) is preferably a poly(ethylene glycol) block/unit typically having a molecular weight of less than about 10,000 per unit. In some embodiments, the molecular weight of the poly(alkylene oxide) is between about 2000 and about 6000 per unit. In some embodiments, the poly(ethylene glycol) block/unit has a molecular weight of 5000 per unit. In some embodiments, the poly(ethylene glycol) block/unit has a molecular weight of less than about 4000 per unit. In some embodiments, the molecular weight is between about 1000 and about 2000 per unit.

Unless otherwise indicated, the molar fractions reported herein are based on the total molar amount of poly(alkylene glycol) and non-glycol units in the polymers.

The term “active agent”, as used herein, encompasses a substance intended for mitigation, treatment, or prevention of disease that stimulates a specific physiologic (metabolic) response. The term also includes an agent for diagnostic purpose such as a contrast agent or a dye. The term also encompasses any substance that possesses structural and/or functional activity in a biological system, including without limitation, organ, tissue or cell based derivatives, cells, viruses, vectors, nucleic acids (animal, plant, microbial, and viral) that are natural and recombinant and synthetic in origin and of any sequence and size, antibodies, polynucleotides, oligonucleotides, cDNA's, oncogenes, proteins, peptides, amino acids, lipoproteins, glycoproteins, lipids, carbohydrates, polysaccharides, lipids, liposomes, or other cellular components or organelles for instance receptors and ligands. Further the term “biological agent”, as used herein, includes virus, serum, toxin, antitoxin, vaccine, blood, blood component or derivative, allergenic product, or analogous product, or arsphenamine or its derivatives (or any trivalent organic arsenic compound) applicable to the prevention, treatment, or cure of diseases or injuries of man (per Section 351(a) of the Public Health Service Act (42 U.S.C. 262(a)). Further the term “biological agent” may include 1) “biomolecule”, as used herein, encompassing a biologically active peptide, protein, carbohydrate, vitamin, lipid, or nucleic acid produced by and purified from naturally occurring or recombinant organisms, antibodies, tissues or cell lines or synthetic analogs of such molecules; 2) “genetic material” as used herein, encompassing nucleic acid (either deoxyribonucleic acid (DNA) or ribonucleic acid (RNA), genetic element, gene, factor, allele, operon, structural gene, regulator gene, operator gene, gene complement, genome, genetic code, codon, anticodon, messenger RNA (mRNA), transfer RNA (tRNA), ribosomal extrachromosomal genetic element, plasmagene, plasmid, transposon, gene mutation, gene sequence, exon, intron, and, 3) “processed biologics”, as used herein, such as cells, tissues or organs that have undergone manipulation. The therapeutic agent may also include vitamin or mineral substances or other natural elements.

The following non-limiting examples set forth herein below illustrate certain aspects of the invention. All parts and percentages are by weight percent unless otherwise noted and all temperatures are in degrees Celsius unless otherwise indicated. All solvents were HPLC grade and all other reagents were of analytical grade and used as received, unless otherwise indicated.

### EXAMPLES

### Example 1

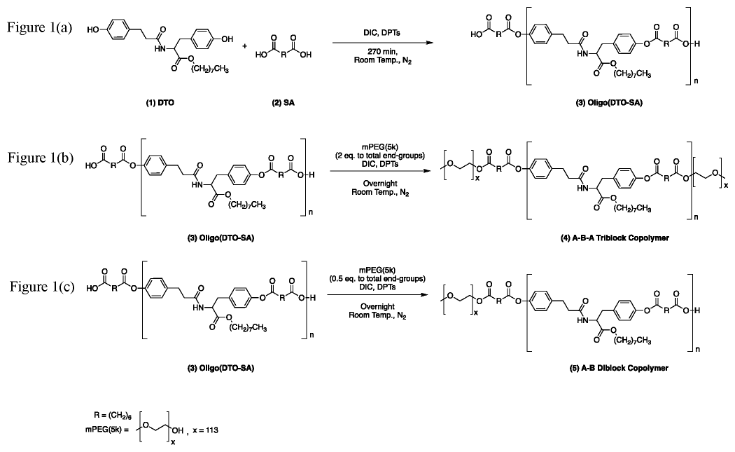

Polymer Synthesis. Oligo(DTO-SA) (B-block), triblock copolymer (A-B-A) and diblock copolymer (A-B-H) were synthesized via one-pot carbodiimide reaction at room temperature and inert atmosphere (FIG. 1). Excess suberic acid was used in the oligo(DTO-SA) reaction to favor the termination of both ends-groups with carboxylic acid and to control polymer growth. The addition of poly(ethylene glycol) methyl ester (mPEG(5k)) to triblock or diblock occurs via esterification reaction between terminal carboxylic acids from oligo(DTO-SA) and the terminal alcohol group from mPEG(5k). The presence of carboxylic acid as end-groups is essential for diblock and triblock formation. Oligo(DTO-SA) synthesis was allowed to go to completion (270 min post first DIC addition), where no significant change in number-average molecular weight (Mn) was observed prior to precipitation (oligo(DTO-SA) isolation) or mPEG(5k) addition (copolymers synthesis (triblock and diblock)). Amount of mPEG(5k) used was calculated to favor the formation of triblock (A-B-A copolymer) or diblock (A-B-H copolymer) structure. For triblock copolymer synthesis excess of mPEG(5k) was used, and this excess was calculated to be twice the theoretical amount required to react with both oligo(DTO-SA) carboxylic acid end-groups. On the other hand, for diblock synthesis, the amount of mPEG(5k) was equivalent to half of the theoretical oligo(DTO-SA) carboxylic acid end-groups.

Procedure for Polymers Synthesis. Desaminotyrosyl-tyrosine octyl ester (DTO) (1.00 eq., 22.65 mmols), suberic acid (SA) (1.10 eq., 24.91 mmols), and 4-dimethylaminopyridinium-p-toluene sulfate (DPTs) (0.54 eq., 12.16 mmols) were added to a flame-dried round bottom flask, and they were dissolved in anhydrous dichloromethane (DCM) (120 mL) at room temperature under nitrogen. After homogenization, N,N′-diisopropylcarbodiimide (DIC) (2.76 eq., 62.60 mmols) was added initiating the hydrophobic oligo(DTO-SA) formation. The reaction was performed under nitrogen, at room temperature, and constant stirring till no change in number-average molecular weight (Mn) was observed by gel permeation chromatography (GPC) (270 min after DIC addition). For Oligo(DTO-SA) synthesis, the polymer was precipitated at this step over IPA ((DCM:IPA 1:5.4 v/v)). For triblock and diblock synthesis, mPEG(5k) addition to the reaction mixture was performed initiating the amphiphilic copolymer formation. Different amounts of mPEG(5k) were added to favor diblock (A-B-H) or triblock (A-B-A) copolymer synthesis. Triblock copolymer synthesis was favored by using an excess amount of mPEG(5k) which corresponds to 2.1 eq. of the theoretical total amount of carboxylic acid end-groups present in the oligo(DTO-SA) reaction mixture. Meanwhile, to favor diblock copolymer synthesis, only 0.5 eq. of mPEG(5k) are used, again based on the theoretical total amount of carboxylic acid end-groups present. An additional amount of DIC (0.51 eq., 11.59 mmols) was introduced into the reaction mixture 5 minutes after addition of mPEG(5k). The reaction was allowed to proceed overnight, under nitrogen, at room temperature and constant stirring to ensure completion. After 24 h of first DIC addition, the reaction mixture was slowly precipitated over isopropanol (IPA) (DCM:IPA 1:5.4 v/v) under vigorous stirring. The turbid solution was refrigerated (10° C.) for 2 hours yielding an oil. The supernatant was decanted, and to the oil, fresh IPA (200 mL) was added, and stirred until a white precipitate formed. The solution was vacuum filtered. All polymers were individually re-dissolved in DCM (50 mL) and re-precipitated over IPA (DCM:IPA 1:5.4 v/v) as previously described to finalize the purification. Final powder was washed with hexanes (200 mL) under stirring. The polymers were isolated by vacuum filtration and dried in a vacuum oven.

### Example 2

Molecular Weight Determination. Final polymer molecular weight, post-purification, were obtained by gel permeation chromatography (GPC) using tetrahydrofuran (THF) as mobile phase (Table 1). Theoretically, each addition of an A-block should yield an increase of 5 kDa in the number-average molecular weight (Mn) once A-block (mPEG(5k)) has Mn of 5 kDa accordantly to the supplier. Even though there is a difference in molecular weight between the synthesized polymers, the expected difference in Mn was not observed. GPC analysis relies on the comparison between the sample hydrodynamic volume and the standard (polystyrene) hydrodynamic volume, and therefore is not an absolute method. Despite identical composition of individual blocks on triblock (A-B-A) and diblock (A-B-H) copolymers they have different hydrophobic-to-hydrophilic ratio, and polystyrene is a hydrophobic polymer. This results in a different interaction of these polymers with the mobile phase. Therefore, GPC cannot be used for structure confirmation in this case, but shows that the synthesized polymers are distinct from each other.

TABLE 1
Summary of polymers molecular weight results obtained by THF-GPC.

| | THF-GPC | | | | |
|---|---|---|---|---|---|
| Polymer | Mn (kDa) | Mw (kDa) | PDI | Sn (kDa) | As |
| mPEG(5k) | 7.24 (0.01) | 7.39 (0.01) | 1.021 (0.000) | 1.1 (0.2) | 1.31 (0.06) |
| Oligo(DTO-SA) | 15.42 (0.05) | 21.78 (0.05) | 1.412 (0.002) | 9.9 (0.9) | 2.38 (0.04) |
| Diblock | 17.96 (0.02) | 23.46 (0.02) | 1.306 (0.000) | 9.9 (0.7) | 2.37 (0.06) |
| Triblock | 21.60 (0.04) | 24.72 (0.08) | 1.143 (0.005) | 8.2 (1.1) | 1.36 (0.05) |

Mn: number-average molecular weight, Mw: weight-average molecular weight, PDI: polydispersity index, Sn: breadth, As: asymmetry factor.
Standard deviation of the independent triplicate is in parenthesis.

Method for Molecular Weight Determination. For final determination of polymer weight-average molecular weight (Mw), number-average molecular weight (Mn), and poly-dispersity index (PDI), polymers samples (dry powder), were dissolved in tetrahydrofuran (THF) (1 mg/mL) and were analyzed by GPC. All solutions were filtrated through a polytetrafluoroethylene (PTFE) (0.45 μm) syringe filter prior to the analysis. THF-GPC was performed using the TOSOH-EcoSEC GPC all in-one GPC system composed of an auto-injector, a dual-pump, column switching valve, column oven, and an IR detector type Bryce or dual flow. TOSOH TSKgel Super HZ2500 and TSKgel Super HZ3000 columns were used in tandem and THF with 250 ppm of BHT was used as mobile phase (0.35 mL/min). Data processing and analysis was realized using EcoSEC GPC Software. Polystyrene was used as standard.

Evaluation of Polymer Peak Shape and Resolution. The most common parameters used to describe a polymer distribution are the number-average molecular weight (Mn), weight-average molecular weight (Mw) and polydispersity index (PDI). However, they don't completely define the distribution. Other parameters such as breadth and skewness are also very important to be known. The breadth is statistically measured as the standard deviation and can be assigned to any polymers where Mn and Mw are known. The standard deviation of Mn (Sₙ) was calculated as described by Rudin A (Table 1).

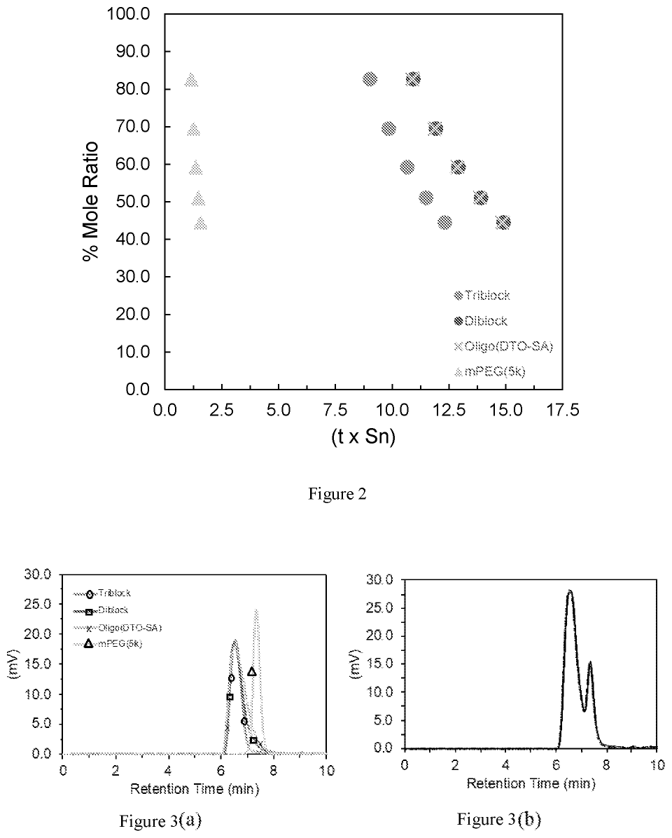

Chebyshev's inequality states that at least (1/t²) of the distribution's values are within the limits of Mn±tSn, where t>1. Approximately 83% of the molecules in the analyzed polymers samples are outside their respective ranges (FIG. 2). mPEG(5k) curve is very close to the abscissa, which indicate low polydispersity. This is also confirmed by PDI obtained by THF-GPC (Table 1). The synthesized polymers on the other hand, are more polydisperse when compared with mPEG(5k) and have similar breadth among each other.

With the number-average molecular weight range stablished it is still important to access the symmetry of a polymer distribution. The asymmetry factor (As) indicates tailing towards low molecular weight (As>1), high molecular weight (As<1), or if the distribution is perfectly symmetric (As=1). Table 1 summarize all asymmetry factors (As) calculated. mPEG(5k) has the most symmetrical distribution. Triblock present a minimal tailing, whereas diblock and oligo(DTO-SA) have similar and more pronounce tailing. All polymers have tailing towards low molecular weight (As>1).

In terms of polymer resolution, GPC was not able to resolve the synthesized polymers as shown in FIG. 3. Only mPEG(5k) was partially resolved. The poor separation of the synthesized polymers, could be explained by the fact that at least 83% of the molecules are about 10 kDa distant from their original Mₙ value (FIG. 2), while the theoretical difference between them are of 5 kDa per mPEG(5k)-block incorporated to the copolymer structure.

Method for Evaluation of Polymers Peak Shape and Resolution. Polymer breadth was performed using the method described by Rudin A (1969) using Mn and Mw data previously obtained by THF-GPC. Asymmetric factor (As) was calculated by TOSOH EcoSEC analysis program. The calculation consists on the division of the distance from the peak maximum to one extreme of the peak (low molecular weight) by the distance from the peak maximum to the other extreme (high molecular weight) both at 10% peak height. Evaluation of peak resolution was performed in two different approaches. First, the final chromatograms obtained for molecular weight determination were simply overlaid. Solutions of all polymer mixed prior to injection on THF-GPC were prepared as an alternative to the first approach. The preparation of the polymer mixture solution was performed by individually dissolving the polymers (triblock, diblock, oligo(DTO-SA) and mPEG(5k)) in THF at 4 mg/mL. Following, 0.5 mL of all four polymers solutions were mixed and the final solution was filter through a PTFE (0.45 μm) syringe filter prior to analysis.

### Example 3

Determination of Free mPEG(5k) Content. Presence of unreacted mPEG(5k) as impurity could be possible even post-purification of triblock and diblock, but minimum amounts would be expected once no pronounced peak corresponding to mPEG(5k) was observed by GPC. By proton-nuclear magnetic resonance (¹H-NMR), the distinction between free and bound mPEG(5k) would only be possible by identifying the end-group peak, an alcohol, or if reacted, an ester. However, end groups of polymers could be challenge to quantify, mainly if other signals from the polymer structure overlaps with the peak of interest. On the other hand, GPC was partially able to resolve mPEG(5k) from the synthesized copolymers (triblock and diblock). Therefore, quantification of free mPEG(5k) was performed via a standard addition calibration curve. Free mPEG(5k) was used as standard, and sample analysis was performed by THF-GPC. Peaks deconvolution was necessary, where peaks were approximated with an exponential modified Gaussian (EMG) function. This function is a combination of a gaussian with an exponential decay function, and is known to better represent chromatographic peaks (symmetrical and asymmetrical). Quantification of free mPEG(5k) present in copolymers samples was obtained by extrapolating the linear regression of the standard addition calibration curve to y=0. For triblock copolymer concentration of free mPEG(5k) was calculated to be 0.017±0.008 mg/mL equivalent to 3.5±1.5% (w/w), and diblock presented 0.067±0.015 mg/mL of free mPEG(5k) present, which is equivalent to 14.9±3.0% (w/w). The amount of free mPEG(5k) calculated to be present on diblock, is higher than the amount calculated for triblock, which is not expected. Diblock synthesis used half the amount necessary to react with all end-groups of oligo(DTO-SA) whereas triblock used excess free mPEG(5k). The calculation of free mPEG(5k) present on diblock could be affected by the pronounce GPC peak tailing towards low molecular weight. This tailing has a significant overlap with free mPEG(5k) peak and deconvolution could be compromised.

Method for Determination of Free mPEG(5k) Content. In order to determine the free mPEG(5k) amount present in each copolymer composition a standard addition calibration curve for each copolymer was prepared by mixing varying amounts of mPEG(5k) as standard (final concentration ranging from 1.0 mg/mL to 0.032 mg/mL) to a fix amount of each individual copolymer (0.5 mg/mL) at constant final volume (2 mL). mPEG(5k) was dissolved in THF and a serial dilution was prepared prior to addition to the copolymer solution. Final solutions were filtered through a PTFE (0.45 μm) syringe filter and the samples were analyzed by TOSOH-EcoSEC THF-GPC. Once both copolymers GPC peaks overlaps with mPEG(5k) peak, deconvolution was performed through an exponential modified Gaussian (Gaussian Mod) approximation using Origin 2018. The deconvolution allowed for mPEG(5k) peak area calculation, which was plotted against the concentration of added mPEG(5k). Extrapolation of the linear regression obtained to y=0 allowed the quantification of free mPEG(5k) present in the initial copolymer sample (without mPEG(5k) addition).

### Example 4

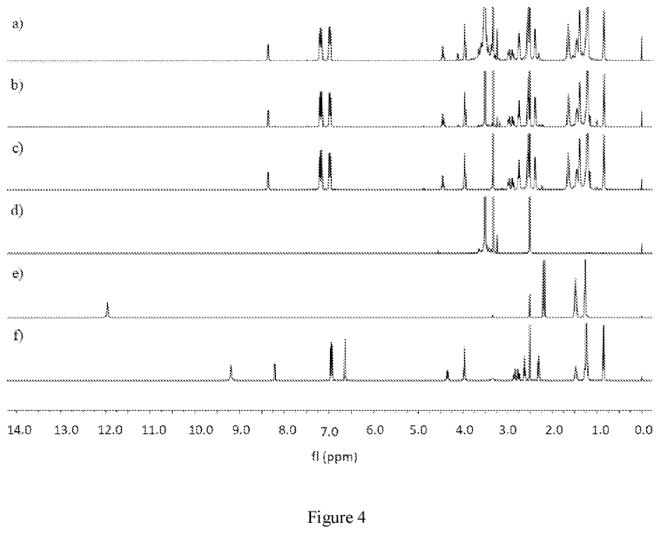

Structural Characterization. As part of the structural characterization of the polymers proton-nuclear magnetic resonance (¹H-NMR) was performed (FIG. 4).

Oligo(DTO-SA) formation was confirmed by comparing ¹H-NMR spectrum of monomers (DTO and suberic acid) with final polymer. Downfield shifts of aromatic peaks from 6.99-6.90 and 6.64 ppm (DTO) to 7.18 and 6.98 ppm (oligo(DTO-SA)), were observed post-polymerization. Also, the shift observed for the protons adjacent to the carbonyl group, 2.19 ppm (suberic acid) to 2.54 ppm (oligo(DTO-SA)), and the one right next to it, 1.56-1.41 (suberic acid) to 1.63 (oligo(DTO-SA)), indicate ester formation between DTO and suberic acid confirming the oligo(DTO-SA) formation. Phenol peak from DTO (9.19 ppm) completely disappeared and traces of carboxylic acid peaks (11.98 ppm) were present post-polymerization. Also, small peak at 2.24 ppm (R—OOC—CH₂CH₂—(CH₂)₂—CH₂CH₂—COOH) and at 1.56-1.50 ppm (R—OOC—CH₂CH₂—(CH₂)₂—CH₂CH₂—COOH) were detected. The peak at 2.24 ppm presented a shift when compared with the suberic acid equivalent peak (2.19 ppm). These observations confirm termination of oligo(DTO-SA) in carboxylic acid in both ends.

Formation of an ester bond between the carboxylic acid (oligo(DTO-SA) end-group) and the alcohol (mPEG(5k) end-group) characterizes both copolymers synthesis (triblock and diblock). This reaction promotes a chemical shift of the protons adjacent to the reactive mPEG(5k) end-group. Initially, in the mPEG(5k) ¹H-NMR spectrum, these protons have a chemical shift of 4.56 ppm, appearing at 4.13-4.09 ppm in the diblock and triblock spectrums. This confirms the reaction between oligo(DTO-SA) and mPEG(5k) towards the copolymers formation. In addition to these observations, diblock copolymer present traces of carboxylic acid proton (11.97 ppm) and other two peaks characteristics of the oligo(DTO-SA) end-group (2.22 ppm, HOOC—CH₂—CH₂—R and 1.56-1.50 ppm HOOC—CH₂—CH₂—R). On the other hand, the 2.22 ppm peak was not observed for triblock.

Triblock. ¹H NMR (500 MHz, DMSO-d₆ with 0.03% (v/v) TMS as reference) δ 8.36 (d, J=7.5 Hz, 1H, NH, DTO), 7.18 (dd, J=17.3, 8.1 Hz, 4H, Ar-H, DTO), 6.98 (dd, J=14.6, 8.0 Hz, 4H, Ar-H, DTO), 4.45 (q, J=7.6, 6.9 Hz, 1H, RNH—CH—R, DTO), 4.12 (t, J=4.6 Hz, OH, CH₃[O—CH₂—CH₂]ₙ—O—CH₂—CH₂—O—R, mPEG(5k)), 3.96 (t, J=6.6 Hz, 2H, R—OOC—CH₂—CH₂—(CH₂)₅—CH₃, DTO), 3.51 (d, 82H, CH₃[O—CH₂—CH₂]ₙ—O—CH₂—CH₂—O—R, mPEG(5k)), 3.32 (H₂O), 3.24 (d, OH, CH₃[O—CH₂—CH₂]ₙ—O—CH₂—CH₂—O—R, mPEG(5k)), 2.98 (dd, J=13.9, 6.2 Hz, 1H, Ar-CH₂—CH(NH—R)—R, DTO), 2.89 (dd, J=13.9, 8.9 Hz, 1H, Ar-CH₂—CH(NH—R)—R, DTO), 2.75 (t, J=7.6 Hz, 2H, Ar-CH₂—CH₂—CO(NH—R), DTO), 2.54 (q, J=7.4 Hz, 4H, R—OOC—CH₂—R, SA), 2.50 (DMSO-d6), 2.45-2.34 (m, 2H, Ar-CH₂—CH₂—CO(NH—R)), 1.74-1.59 (m, 4H, R—OOC—CH₂—CH₂—R, SA), 1.59-1.50 (m, OH, HOOC—CH₂—CH₂—R, SA end-group), 1.45 (t, J=6.7 Hz, 2H, R—OOC—CH₂—CH₂—(CH₂)₅—CH₃, DTO), 1.42-1.31 (m, 4H, R-CH₂—CH₂—CH₂—COOH, SA), 1.22 (s, 11H, R—OOC—CH₂—CH₂—(CH₂)₅—CH₃, DTO), 0.84 (t, J=6.9 Hz, 3H, R—OOC—CH₂—CH₂—(CH₂)₅—CH₃, DTO), 0.00 (TMS).

Diblock. ¹H NMR (500 MHz, DMSO-d₆ with 0.03% (v/v) TMS as reference) δ 11.97 (s, OH, COOH, SA end-group), 8.35 (d, J=7.6 Hz, 1H, NH, DTO), 7.18 (dd, J=17.4, 8.1 Hz, 4H, Ar-H, DTO), 6.98 (dd, J=14.9, 8.2 Hz, 4H, Ar-H, DTO), 4.87 (p, J=6.2 Hz, OH,), 4.52-4.38 (m, 1H, RNH—CH—R, DTO), 4.13-4.09 (m, OH, CH₃—[O—CH₂—CH₂]ₙ—O—CH₂—CH₂—O—R, mPEG(5k)), 3.96 (t, J=6.5 Hz, 2H, R—OOC—CH₂—CH₂—(CH₂)₅—CH₃, DTO), 3.51 (s, 12H, CH₃[O—CH₂—CH₂]ₙ—O—CH₂—CH₂—O—R, mPEG(5k)), 3.32 (H₂O), 3.24 (s, OH, CH₃—[O—CH₂—CH₂]ₙ—O—CH₂—CH₂—O—R, mPEG(5k)), 3.05-2.93 (m, 1H, Ar-CH₂—CH(NH—R)—R, DTO), 2.88 (dd, J=13.9, 8.9 Hz, 1H, Ar-CH₂—CH(NH—R)—R, DTO), 2.74 (t, J=7.6 Hz, 2H, Ar-CH₂—CH₂—CO(NH—R), DTO), 2.54 (t, J=7.1 Hz, 4H, R—OOC—CH₂—R, SA), 2.50 (DMSO-d6), 2.38 (t, J=7.7 Hz, 2H, Ar-CH₂—CH₂—CO(NH—R)), 2.22 (dt, J=18.5, 7.3 Hz, OH, HOOC—CH₂—CH₂-R, SA end-group), 1.63 (h, J=7.2 Hz, 4H, R—OOC—CH₂—CH₂-R, SA), 1.56-1.50 (m, OH, HOOC—CH₂—CH₂-R, SA end-group), 1.45 (t, J=6.8 Hz, 2H, R—OOC—CH₂—CH₂—(CH₂)₅—CH₃, DTO), 1.39 (q, J=4.2 Hz, 4H, R-CH₂—CH₂—CH₂—COOH, SA), 1.29-1.17 (m, 11H, R—OOC—CH₂—CH₂—(CH₂)₅—CH₃, DTO), 0.84 (t, J=6.7 Hz, 3H, R—OOC—CH₂—CH₂—(CH₂)₅—CH₃, DTO), 0.00 (TMS).

mPEG(5k). ¹H NMR (500 MHz, DMSO-d₆ with 0.03% (v/v) TMS as reference) δ 4.56 (td, J=5.5, 0.8 Hz, 1H, CH₃[O—CH₂—CH₂]ₙ—O—CH₂—CH₂—OH), 3.51 (d, J=0.9 Hz, 452H, CH₃[O—CH₂—CH₂]ₙ—O—CH₂—CH₂—OH), 3.32 (H₂O), 3.24 (d, J=0.8 Hz, 3H, CH₃—[O—CH₂—CH₂]ₙ—O—CH₂—CH₂—OH), 2.50 (DMSO-d6), 0.00 (TMS).

Oligo(DTO-SA). ¹H NMR (500 MHz, DMSO-d₆ with 0.03% (v/v) TMS as reference) δ 11.98 (s, OH, COOH, SA end-group), 8.36 (d, J=7.5 Hz, 1H, NH, DTO), 7.18 (dd, J=17.4, 8.1 Hz, 4H, Ar-H, DTO), 6.98 (dd, J=14.7, 8.0 Hz, 4H, Ar-H, DTO), 4.88 (p, J=6.3 Hz, OH), 4.52-4.38 (m, 1H, Ar-CH₂—CH(NH—R)—R, DTO), 4.06-3.88 (m, 2H, R—OOC—CH₂—CH₂—(CH₂)₅—CH₃, DTO), 3.32 (H₂O), 2.98 (dd, J=13.9, 6.1 Hz, 1H, Ar-CH₂—CH(NH—R)—R, DTO), 2.89 (dd, J=13.9, 8.8 Hz, 1H, Ar-CH₂—CH(NH—R)—R, DTO), 2.74 (t, J=7.6 Hz, 2H, Ar-CH₂—CH₂—CO(NH—R), DTO), 2.54 (q, J=6.9, 6.4 Hz, 4H, R—OOC—CH₂-R, SA), 2.50 (DMSO-d6), 2.38 (t, J=7.4 Hz, 2H, Ar-CH₂—CH₂—CO(NH—R), DTO), 2.24 (t, J=7.4 Hz, OH, HOOC—CH₂—CH₂-R, SA end-group), 1.63 (h, J=7.7, 7.3 Hz, 4H, R—OOC—CH₂—CH₂-R, SA), 1.56-1.50 (m, OH, HOOC—CH₂—CH₂—R, SA end-group), 1.45 (t, J=6.8 Hz, 2H, R—OOC—CH₂—CH₂—(CH₂)₅—CH₃, DTO), 1.39 (d, J=6.2 Hz, 4H, R-CH₂—CH₂—CH₂—COOH, SA), 1.22 (s, 12H, R—OOC—CH₂—CH₂—(CH₂)₅—CH₃, DTO), 0.84 (t, J=6.7 Hz, 3H, R—OOC—CH₂—CH₂—(CH₂)₅—CH₃, DTO), 0.00 (TMS).

DTO. ¹H NMR (500 MHz, DMSO-d₆ with 0.03% (v/v) TMS as reference) δ 9.19 (s, 2H, Ar-OH), 8.22 (d, J=7.7 Hz, 1H, NH), 6.99-6.90 (m, 4H, Ar—H), 6.64 (ddd, J=8.3, 4.4, 1.1 Hz, 4H, Ar—H), 4.39-4.31 (m, 1H, Ar-CH₂—CH(NH—R)—R), 4.00-3.92 (m, 2H, R—OOC—CH₂—CH₂—(CH₂)₅—CH₃), 3.33 (H₂O), 2.85 (dd, J=13.8, 6.1 Hz, 1H, Ar-CH₂—CH(NH—R)—R, 2.75 (dd, J=13.8, 8.8 Hz, 1H, Ar-CH₂—CH(NH—R)—R), 2.62 (dd, J=9.0, 6.7 Hz, 2H, Ar-CH₂—CH₂—CO(NH—R), 2.50 (DMSO-d6), 2.30 (dd, J=8.9, 6.9 Hz, 2H, Ar-CH₂—CH₂—CO(NH—R)), 1.47 (dd, J=9.7, 4.4 Hz, 2H, R—OOC—CH₂—CH₂—(CH₂)₅—CH₃), 1.23 (s, 12H, R—OOC—CH₂—CH₂—(CH₂)₅—CH₃), 0.88-0.82 (m, 3H, R—OOC—CH₂—CH₂—(CH₂)₅—CH₃), 0.00 (TMS).

Suberic Acid. ¹H NMR (500 MHz, DMSO-d6 with 0.03% (v/v) TMS as reference) δ 11.96 (s, 2H, R—COOH), 3.34 (H₂O), 2.50 (DMSO-d6), 2.19 (td, J=7.4, 1.0 Hz, 4H, R—CH₂—CH₂—CH₂COOH), 1.56-1.41 (m, 4H, R—CH₂CH₂CH₂COOH), 1.26 (p, J=3.7 Hz, 4H, R—CH₂CH₂CH₂COOH), 0.00 (TMS).

Method for Structural Analysis. Structural analysis of the synthesized copolymers was realized by proton nuclear magnetic resonance (′H-NMR) spectroscopy. The analysis was performed in deuterated dimethyl sulfoxide (DMSO-d6) with tetramethylsilane (TMS) (0.03% v/v) as internal standard using a Varian VNMRS 500 MHz spectrophotometer where 128 scans were collected. NMR spectrums were analyzed using MestReNova software version 8.0.1-10878.

The following polymers have also been prepared using procedures described herein. T and D represent triblock and deblock, respectively.

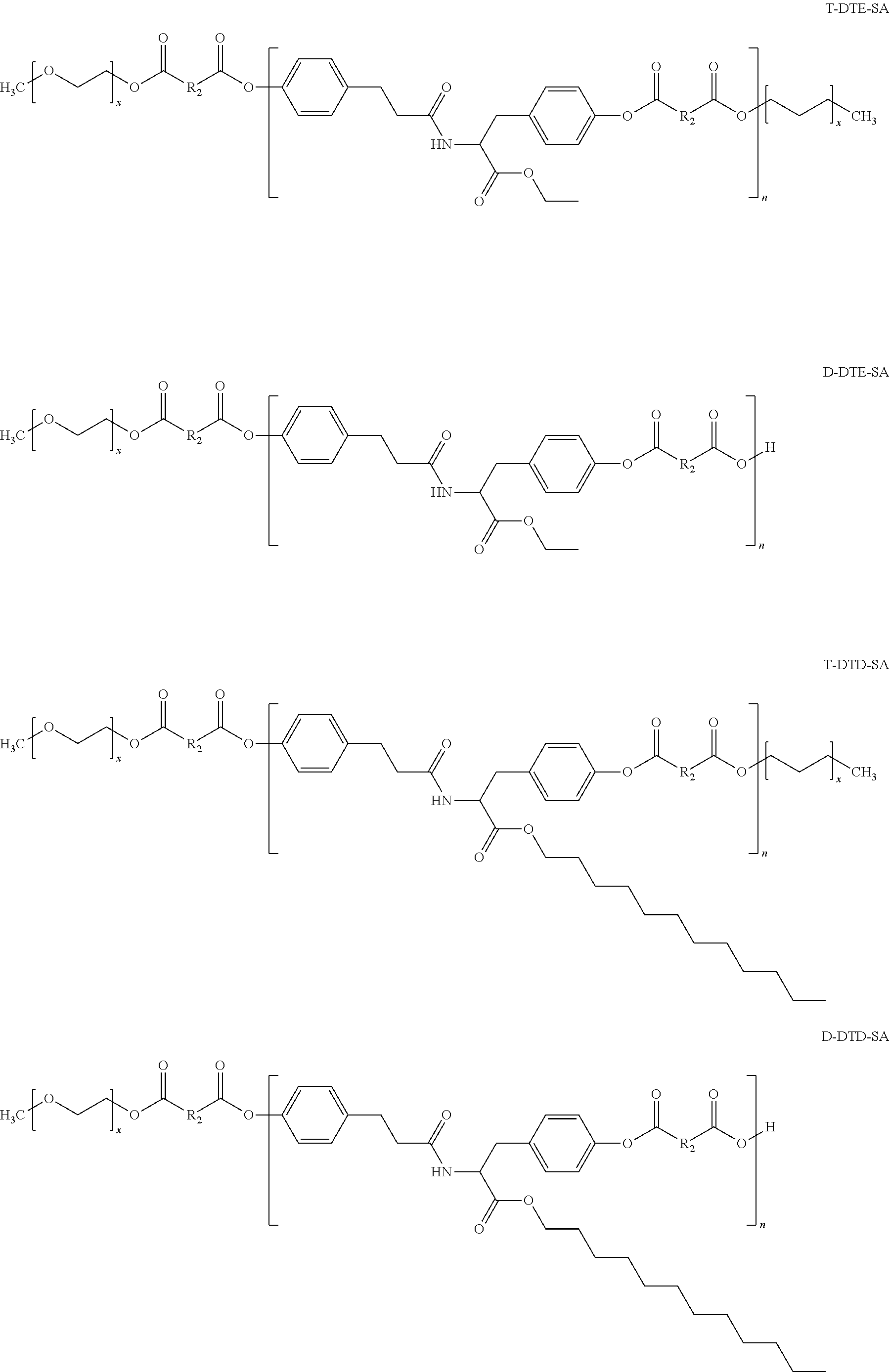

### Example 5

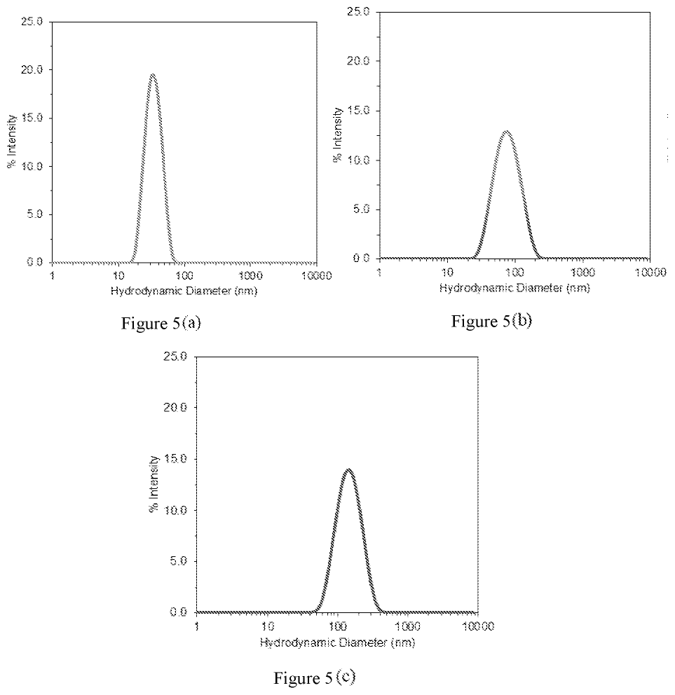

Single Copolymer Nanospheres. Synthesized triblock and diblock copolymers are both amphiphilic macromolecules that self-assemble in aqueous media forming nanospheres with a core-shell structure. Size analysis was performed by dynamic light scattering (DLS) (FIG. 5). Both copolymers presented a monodisperse distribution (PDI<0.2), but distinct Z-average hydrodynamic diameter, being triblock (Z-Average hydrodynamic diameter=32.8±0.7 nm) smaller than diblock (Z-Average hydrodynamic diameter=129.3±2.3 nm). Percent polymer recover post nanospheres preparation was calculated to be 58±4% for triblock and 65±7% for diblock.

Method for Single Copolymer Nanospheres Preparation. For nanospheres preparation the tyrosine-based copolymer (60 mg) was dissolved in dimethyl formamide (DMF) (0.1 mg/mL) and it was precipitated drop-wise into 14.5 mL of phosphate buffered saline (PBS) (pH 7.4) under mild stirring at room temperature, yielding a turbid suspension. The suspension was filtered through a polyvinylidene fluoride (PVDF) (0.22 μm) syringe filter and it was ultra-centrifuged at 65000 rotations per minute (rpm), for 3 hours at 18° C. using a Beckman Coulter Optima™ L-90K ultracentrifuge. Following, the supernatant was discarded, the pellet was washed twice (1 mL PBS/wash), and it was re-suspended in PBS (1 mL) overnight under stirring (200 rpm) using an orbital-shaker at room temperature. The preparation was performed for both copolymers in independent triplicate. To each vial, 100 μL of the respective solution (nanospheres resuspension or PBS) was added, and the vials were freeze-dried overnight. Following, the vials were re-weighted in triplicate. Total solids mass (mg) was obtained by the difference between the average of full vial mass and the average of the empty vial mass. PBS salts mass was subtracted from the total solid mass yielding polymer mass (mg). Percent polymer yield was then obtained by correlating the polymer mass recovered post-nanospheres preparation to the initial amount of polymer used for the nanospheres preparation.

Nanospheres Size Analysis. Nanospheres Z-average hydrodynamic diameter was obtained via dynamic light scattering (DLS) at 25° C. using a Class I laser on a Malvern Zetasizer Nano S particle size analyzer. Particle size analysis, was performed by Malvern Zetasizer Software (version 7.12). Brownian motion of spherical particles was assumed for Z-average hydrodynamic diameter calculation using Stokes-Einstein equation.

### Example 6

### Multiple Polymeric Components Nanospheres:

Copolymers Blend Nanospheres. Nanospheres size control was achieved by blending triblock and diblock copolymers at different ratios prior to precipitation in aqueous media. The distribution shifts towards larger sizes (to the right) with the increase in diblock on polymer blend composition (FIG. 5). Also, nanospheres prepared from copolymer blends have similar polydispersity index to the nanospheres made of a single copolymer and are monodisperse distributions (PDI<0.2). These indicate that diblock and triblock co-assembles instead of forming an heterogenous mixture of diblock (large) and triblock (small) particles. The correlation of size to blend composition is shown in FIG. 6. Even, small changes in composition (3.33% increase in diblock) results in a change on the Z-average of the hydrodynamic diameter of the nanospheres. The correlation does not follow a linear response. Percent polymer recover post nanospheres preparation was calculated to be in between 61±5% (T:D 58:2, mg:mg) and 78±2% (T:D 20:40 mg:mg) for all blends of diblock with triblock tested.

Table 2 illustrates the size of nanoparticles formed from different combinations of triblock and diblock copolymers.

TABLE 2
Nanoparticles formed from triblock and diblock copolymers.

| | DLS | | | | | | | |
|---|---|---|---|---|---|---|---|---|
| | | | | | | | | St. Dev |
| | | | | | | | | of the Peak |
| Triblock | Diblock | % | Z-Average Size | | Average PDI | Peak Size | | Distribution |
| (mg) | (mg) | Diblock | (nm) (n = 3) | | (n = 3) | (d · nm) (n = 3) | | (d · nm) (n = 3) |
| 60 | 0 | 0.0 | 32.8 | (0.7) | 0.063 (0.013) | 35.3 | (0.5) | 9.7 (0.3) |
| 58 | 2 | 3.3 | 35.0 | (0.3) | 0.058 (0.012) | 37.6 | (0.3) | 10.2 (0.3) |
| 55 | 5 | 8.3 | 39.2 | (0.5) | 0.087 (0.008) | 43.3 | (0.6) | 13.5 (0.5) |
| 50 | 10 | 16.7 | 52.8 | (1.8) | 0.112 (0.058) | 61.8 | (3.4) | 24.8 (3.0) |
| 45 | 15 | 25.0 | 67.8 | (1.3) | 0.159 (0.009) | 81.4 | (2.3) | 34.5 (1.5) |
| 40 | 20 | 33.3 | 77.2 | (2.2) | 0.150 (0.010) | 91.5 | (2.8) | 38.2 (1.6) |
| 35 | 25 | 41.7 | 91.3 | (1.9) | 0.144 (0.013) | 107.3 | (3.7) | 42.0 (3.0) |
| 30 | 30 | 50.0 | 97.2 | (2.0) | 0.137 (0.008) | 112.9 | (2.6) | 43.7 (0.3) |
| 25 | 35 | 58.3 | 101.6 | (1.2) | 0.132 (0.007) | 118.3 | (1.2) | 46.1 (0.6) |
| 20 | 40 | 66.7 | 107.4 | (1.6) | 0.116 (0.020) | 122.6 | (2.8) | 45.9 (1.7) |
| 15 | 45 | 75.0 | 112.8 | (1.1) | 0.114 (0.012) | 128.5 | (2.0) | 45.9 (1.4) |
| 10 | 50 | 83.3 | 117.3 | (1.1) | 0.113 (0.013) | 133.2 | (1.9) | 47.6 (2.1) |
| 5 | 55 | 91.7 | 120.5 | (0.9) | 0.123 (0.008) | 126.4 | (9.8) | 52.1 (1.2) |
| 0 | 60 | 100.0 | 129.3 | (2.3) | 0.158 (0.035) | 149.9 | (12.6) | 59.4 (9.5) |

Experiment was performed in triplicate. In parenthesis is the standard deviation of the triplicate.

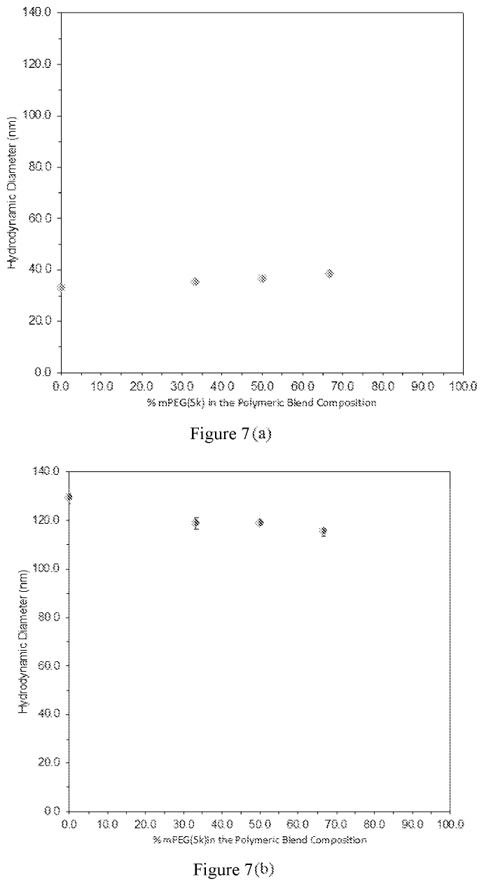

mPEG(5k) and Copolymers Blend Nanospheres. mPEG(5k) causes slight variation on nanospheres Z-average hydrodynamic diameter. For blends composed of mPEG(5k) and triblock the nanospheres size slightly increases with the increase of % mPEG(5k) (FIG. 7). In this case the Z-average hydrodynamic diameter ranged from 32.8±0.7 nm (100% triblock) to 38.3±0.4 nm (66.7% mPEG(5k)) (a). Percent polymer recover post nanospheres preparation was calculated to be 42±1% for the blend initially containing 33.3% mPEG(5k) and 66.7% triblock; and 23±2% for the blend initially containing 66.7% mPEG(5k) and 33.3% triblock. On the other hand, for blends composed of mPEG(5k) and diblock the nanospheres size slightly decreases with the increase of % mPEG(5k). Z-average hydrodynamic diameter ranged from 129.3±2.3 nm (100% diblock) to 115.3±1.6 nm (66.7% mPEG(5k)) (b). Percent polymer recover post nanospheres preparation was calculated to be 52±1% for the blend initially containing 33.3% mPEG(5k) and 66.7% diblock; and 26±3% for the blend initially containing 66.7% mPEG(5k) and 33.3% diblock. This small variation in size observed for mixtures containing mPEG(5k) data confirms that even if free mPEG(5k) is present in the triblock and diblock composition (as indicated by the GPC chromatogram deconvolution), mPEG(5k) would not be the specie responsible for the nanospheres size variation observed when the two copolymers (triblock and diblock) are blended together. Also, the percent polymer recovered post nanospheres preparation drastically decrease with the increase of mPEG(5k) content on the blend with both copolymers. This indicates that mPEG(5k) is lost during the preparation, which is expected due to the hydrophilicity of this polymer.

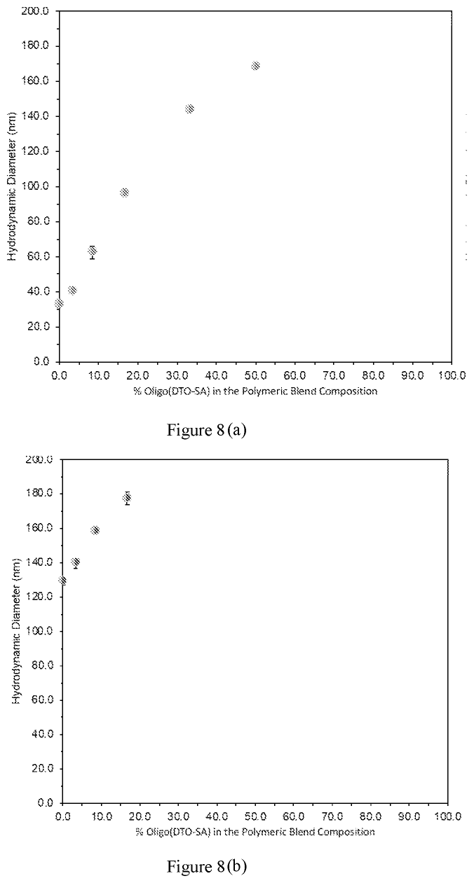

Oligo(DTO-SA) and Copolymers Blend Nanospheres. Nanospheres size also increased by blending oligo(DTO-SA) with a copolymer (triblock or diblock) to a certain limit (FIG. 8). When triblock copolymer was mixed with oligo(DTO-SA), nanospheres Z-average hydrodynamic diameter ranged from 32.8 nm±0.7 nm (100% triblock) to 168.4±1.9 nm (50% oligo(DTO-SA)) (a). Percent polymer recover post nanospheres preparation was calculated to be in between 60±2% (oligo(DTO-SA):Triblock 2:58, mg:mg); and 63±3% (oligo(DTO-SA):Triblock 10:50, mg:mg) for all successful blends containing oligo(DTO-SA) and triblock. Whereas when oligo(DTO-SA) was mixed with diblock copolymer the Z-average hydrodynamic diameter ranged from 129.3±2.3 nm (100% diblock) to 177.6±3.9 nm (16.7% oligo(DTO-SA)) (b). Percent polymer recover post nanospheres preparation was calculated to be in between 68±4% (oligo(DTO-SA):Diblock 10:50, mg:mg); and 78±5% (oligo(DTO-SA):Diblock 5:55, mg:mg) for all successful blends containing oligo(DTO-SA) and diblock. Precipitation prior to ultracentrifugation step was observed for some compositions tested: Oligo(DTO-SA):Triblock (mg:mg): 40:20; and Oligo(DTO-SA):Diblock (mg:mg): 20:40, 30:30 and 40:20. It is hypothesized that oligo(DTO-SA) is incorporated into the core of nanospheres when mixed with a copolymer (diblock or triblock). The hydrophobic character of oligo(DTO-SA) favors the interaction with the nanospheres hydrophobic core formed via the self-assembly of the amphiphilic copolymer (triblock or diblock). Once nanosphere core maximum capacity is reached middle block precipitate out in aqueous media (hydrophilic environment) and the particles are no longer stable.

Method for Multiple Polymeric Components Nanospheres Preparation. The same procedure described for empty nanospheres preparation was realized for the multi-component nanospheres in independently triplicate, but instead of using only one copolymer different polymers were blended in different ratios prior to dissolution in DMF. Final polymer mass was always kept at 60 mg and the amount of DMF used was also kept at 0.6 mL/sample. Following are the tested combinations and their respective ratios: a) Copolymers Blend Nanospheres (Diblock:Triblock (mg:mg): 2:58, 5:55, 10:50, 15:45, 20:40, 25:35, 30:30, 35:25, 40:20, 45:15, 50:10, 55:5); b) mPEG(5k) and Triblock Blend Nanospheres (mPEG(5k):Triblock (mg:mg): 20:40, 30:30, 40:20); c) mPEG(5k) and Diblock Blend Nanospheres (mPEG(5k):Diblock (mg:mg): 20:40, 30:30, 40:20); d) Oligo(DTO-SA) and Triblock Blend Nanospheres (Oligo(DTO-SA):Triblock (mg:mg): 2:58, 5:55, 10:50, 20:40, 30:30, 40:20); e) Oligo(DTO-SA) and Diblock Blend Nanospheres (Oligo(DTO-SA):Diblock (mg:mg): 2:58, 5:55, 10:50, 20:40, 30:30, 40:20).

Nanospheres Size Analysis. Nanospheres Z-average hydrodynamic diameter was obtained via dynamic light scattering (DLS) at 25° C. using a Class I laser on a Malvern Zetasizer Nano S particle size analyzer. Particle size analysis, was performed by Malvern Zetasizer Software (version 7.12). Brownian motion of spherical particles was assumed for Z-average hydrodynamic diameter calculation using Stokes-Einstein equation.

It will be understood by those skilled in the art that numerous and various modifications can be made without departing from the spirit of the present invention. Therefore, the various embodiments and examples of the present invention described herein are illustrative only and not intended to limit the scope of the present invention.

1. A nanosphere composition for delivery of an active agent, comprising a distribution of nanospheres with essentially the same hydrodynamic Z-average diameter in a pharmaceutically acceptable carrier, said nansospheres consisting essentially of a mixture of the same triblock oligomer and the same diblock oligomer, wherein:
the triblock oligomer consists of a single A-B-A structure and the diblock oligomer consists of a single A-B-H structure, wherein A and B in the diblock oligomer are identical to A and B in the triblock oligomer;
wherein the B block is hydrophobic with repeating units having the structure according to Formula I: 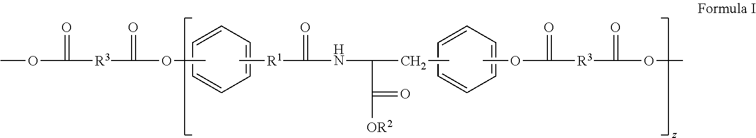
wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms, wherein R² and R³ together contain more than 6 carbons, provided that when R² is (CH₂)₃CH₃, R³ is not (CH₂)₄;
wherein the A block is a poly(alkylene oxide) having the structure: 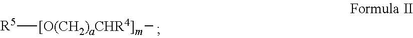
R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
m for each A is independently selected to provide a molecular weight for each A between about 1000 and about 15,000 g/mol.

2. The composition of claim 1, wherein the A block has the structure:
CH₃O—[CH₂—CH₂—O—]ₘ.

3. The composition of claim 1, wherein R¹ is —CH₂—CH₂—.

4. The composition of claim 1, wherein R² is selected from the group consisting of ethyl, butyl, hexyl, octyl, decyl, dodecyl and benzyl groups.

5. The composition of claim 1, wherein R³ contains up to 12 carbon atoms.

6. The composition of claim 1, wherein R³ is selected from the group consisting of —CH₂—CH₂—C(═O)—, —CH═CH—, —CH₂—CH(—OH)—, —CH₂—C(═O)— and (—CH₂—)_{Y}, wherein Y is between 0 and 12, inclusive.

7. The composition of claim 1, wherein the diblock oligomer is at least 30% of the total weight of the diblock oligomer and the triblock oligomer.

8. The composition of claim 1, wherein the diblock oligomer ranges from about 40% to about 99% of the total weight of the diblock oligomer and the triblock oligomer.

9. The composition of claim 1, wherein the nanospheres enclose a pharmaceutically active hydrophobic compound.

10. The composition of claim 1, wherein the pharmaceutically active hydrophobic compound is selected from the group consisting of anti-tumor agents, antibiotics, antimicrobials, statins, peptides, proteins, hormones, and vaccines.

11. The composition of claim 1, wherein the hydrophobic compound is selected from the group consisting of paclitaxel, camptothecin, 9-nitrocamptothecin, cisplatin, carboplatin, ciprofloxacin, doxorubicin, rolipram, simvastatin, methotrexate, indomethacin, probiprofen, ketoprofen, iroxicam, diclofenac, cyclosporine, etraconazole, rapamycin, nocodazole, colchicine, ketoconazole, tetracycline, minocycline, doxycycline, ofloxacin, gentamicin, octreotide, calcitonin, interferon, testosterone, progesterone, estradiol, estrogen, and insulin.

12. The composition of claim 1, wherein the nanospheres enclose a contrast agent.

13. The composition of claim 1, wherein the hydrodynamic Z-average diameter ranges from about 30 nm to about 130 nm.

14. A method of preparing nanoparticles having a predetermined hydrodynamic Z-average diameter as measured by DLS, consisting essentially of triblock oligomers having the same A-B-A structure and diblock oligomers having the same A-B-H structure, wherein A and B in the diblock oligomer are identical to A and B in the triblock oligomer, the method comprising:
(a) blending separate quantities of the triblock and diblock oligomers, wherein the respective quantities are selected to provide the nanoparticles having a predetermined hydrodynamic Z-average diameter,
(b) dissolving the blended oligomers in an organic solvent in which the oligomers are soluble to provide an organic solution of the diblock and triblock oligomers, and
(c) adding the organic solution to an aqueous solution to form an aqueous suspension of the nanoparticles having a predetermined hydrodynamic Z-average diameter;
wherein the B block is hydrophobic with repeating units having the structure according to Formula I: 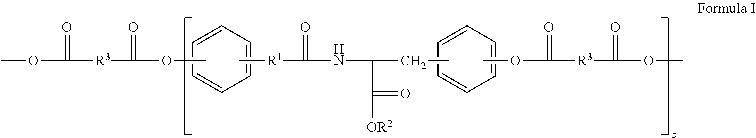
wherein
Z is an integer, between 2 and about 100, inclusive, that provides the B block with a weight-average molecular weight between about 1000 and about 30,000 g/mol;
R¹ is CH═CH or (CH₂)ₙ wherein n is from 0 to 18, inclusive;
R² is straight or branched alkyl and alkylaryl groups containing up to 18 carbon atoms;
R³ is selected from the group consisting of a bond or straight and branched alkyl and alkylaryl groups containing up to 18 carbon atoms, wherein R² and R³ together contain more than 6 carbons;
wherein the A block is a poly(alkylene oxide) having the structure: 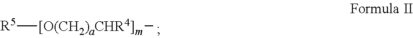
R⁴ for each A and within each A is independently selected from the group consisting of hydrogen and lower alkyl groups containing from one to four carbon atoms;
R⁵ for each A and within each A is independently selected from the group consisting of hydrogen and lower alkyl groups containing from one to four carbon atoms;
m for each A is independently selected to provide a molecular weight for each A between about 1000 and about 15,000 g/mol.

15. The method of claim 14, wherein the diblock oligomer ranges from about 40% to about 90% in the total weight of the triblock oligomer and the diblock oligomer.

16. The method of claim 14, wherein the average hydrodynamic Z-average diameter of the nanospheres range from about 35 nm to about 130 nm.

17. The method of claim 14, wherein the average hydrodynamic Z-average diameter of the nanospheres range from about 50 nm to about 120 nm.

18. The method of claim 14, further comprising mixing a hydrophobic compound with the triblock oligomer and the diblock oligomer.

19. The method of claim 14, wherein the hydrophobic compound is selected from the group consisting of anti-tumor agents, antibiotics, antimicrobials, statins, peptides, proteins, hormones, and vaccines.

20. A method for site-specific or systemic drug delivery comprising administering to a subject in need thereof the composition of claim 1.

21-24. (canceled)
